(19) United States
(12) Patent Application Publication
(10) Pub. No.: US 2020/0390896 A1
(43) Pub. Date: Dec. 17, 2020

(54) Polyamine Prodrugs And Polyamine Prodrug Formulations

(71) Applicant: BOARD OF REGENTS OF THE UNIVERSITY OF NEBRASKA, Lincoln, NE (US)

(72) Inventor: David Oupicky, La Vista, NE (US)

(21) Appl. No.: 15/733,574

(22) PCT Filed: Mar. 6, 2019
(86) PCT No.: PCT/US19/20909

§ 371 Date: Sep. 1, 2020

Related U.S. Application Data
(60) Provisional application No. 62/639,215, filed on Mar. 6, 2018.

(51) Int. Cl. A61K 47/64; A61K 31/785; A61K 31/765; A61P 35/00

(52) U.S. Cl. CPC A61K 47/64; A61K 31/785; A61K 45/06; A61P 35/00; A61K 31/765

(57) ABSTRACT

Provided herein are copolymers comprising monomers of a polyamine and a degradable linker, and further comprising a stabilizing moiety, a fluorinated moiety, a poly(ethylene glycol) (PEG) which is optionally substituted with a targeting moiety, or a combination thereof. Also provided are nanoparticles comprising copolymers as described herein, and methods of using the copolymers and nanoparticles for treating diseases or disorders, e.g., Snyder Robinson Disease or cancer.

### BACKGROUND

Synthetic delivery vectors based on self-assembly of nucleic acids and polycations (polyplexes) continue to gain strength as viable alternatives to viral vectors. Significant effort has been devoted to the synthesis of safe and efficient biodegradable polycations. The polymers of the present invention are bioreducible polycations (BRPs) having the benefits of reduced toxicity compared to non-degradable polycations and better spatial control of polyplex disassembly compared to hydrolytically degradable polycations. Improved spatial control of polyplex disassembly and release of DNA that is localized predominantly to the cytoplasm and nucleus have been shown to enhance transfection of several types of nucleic acids (plasmid DNA, mRNA, siRNA, microRNA) in a number of cancer cell lines. Bioreducible polycations are degraded selectively in the reducing intracellular space (Christensen et al., Bioconjugate Chem, (2006) 17: 1233-1240, Zhang et al., J Controlled Rel, (2010) 143: 359-366). The degradation is mediated by thiol/disulfide exchange reactions with small redox molecules like glutathione (GSH); possibly with the help of redox enzymes (Biaglow et al., Anal Biochem, (2000) 281: 77-86). GSH is the most abundant intracellular thiol present in millimolar concentrations inside the cell but only in micromolar concentrations in the blood plasma (Jones et al., Clin Chim Acta, (1998) 275: 175-84). The majority of GSH is usually found in the cytoplasm (1-11 mM), which is also the principal site of GSH biosynthesis. The most reducing environment is usually found within the nucleus, where it is required for DNA synthesis and repair and to maintain a number of transcription factors in reduced state. Metastatic cancer cells have been shown to have significantly elevated levels of GSH. BRPs are thus particularly promising for nucleic acid delivery to metastatic cancers because significantly elevated levels of GSH are often associated with high metastatic potential of cells.

Polyamines are found in both eukaryotic and prokaryotic cells and figure prominently in regulation of the cell cycle and cell division. Agents specifically targeting polyamine biosynthesis, such as polyamine analogs, have been shown to have therapeutic effect in treatment of cancer, parasitic diseases, and other indications. MicroRNAs (miRNAs) show promise as cancer therapeutics because they can simultaneously affect multiple oncogenic pathways. Therapies that combine nucleic acids with small-molecule drugs, such as polyamines, have the potential to greatly enhance the treatment repertoire and efficacy for many diseases, including cancer. Despite the tremendous potential, the relatively large size and highly charged nature of nucleic acids such as miRNA presents a major unsolved pharmaceutical challenge in achieving successful systemic therapeutic delivery and efficacious treatment. A need exists to address these challenges.

### SUMMARY

Provided herein are copolymers comprising monomers of (1) a polyamine and (2) a degradable linker, and further comprising a stabilizing moiety, a fluorinated moiety, a poly(ethylene glycol) (PEG) which is optionally substituted with a targeting moiety, or a combination thereof. More particularly, provided are nanoparticles comprising the copolymers and a nucleic acid, and the uses of such nanoparticles in treating diseases or disorders, e.g., to treat cancer, Snyder Robinson Disease, and other conditions associated with altered cellular polyamine metabolism.

In one aspect, the disclosure provides copolymers (PaPs) comprising PG-11047 (PaP-P), BENSpm (PaP-B), or spermine. In another aspect, the disclosure provides copolymers comprising bis(2-hydroxyethyl)disulfide (BHED).

Further provided herein are methods of using the copolymers disclosed to treat or prevent a Snyder Robinson Disease and cancer in a subject. In one aspect, the use comprises administering to a subject a nanoparticle comprising a copolymer disclosed herein and a nucleic acid, e.g., a miRNA. In some aspects, the miRNA comprises miR34a.

Other aspects of the disclosure include a copolymer or nanoparticle as disclosed herein for use in the preparation of a medicament for treating or preventing a disease or disorder in a subject, and the use of a compound as disclosed herein in a method of treating or preventing a disease or disorder in a subject.

### BRIEF DESCRIPTION OF THE FIGURES

FIG. 1 shows that copolymers used to assemble nanoparticle containing miR34a provide improved anticancer activity in colorectal xenograft in mice.

FIG. 2 shows characterization of PaPs and PaP/miRNA nanoparticles. (a) Kinetics of PaP activation and release of BENSpm and PG11047 in the presence of 10 mM DTT analyzed by NMR and HPLC. (b) Gel retardation assay to assess PaP condensation and loading of miRNA in PaP-B/miRNA nanoparticles. (c) Size of PaP-B/miRNA particles measured by DLS and TEM (bar=50 nm). (d) Reduction-triggered disassembly of PaP-B/miRNA particles and release of miRNA. PaP/miRNA were incubated +/−10 mM GSH and increasing concentrations of heparin. Release of miRNA was measured by agarose gel electrophoresis. (e) Effect of simulated intracellular reducing conditions (10 mM GSH) on the size and morphology of PaP/miRNA particles assessed by DLS (top) and TEM (bottom, bar=200 nm). (f) FACS analysis of the uptake of PaP/FAM-miRNA in HCT116 cells (4 h inc., 200 nM miRNA, mean fluorescence per cell +/−SD, n=3).

FIG. 3 shows the effect of PaPs and PaP/miRNA particles on polyamine catabolism. (a) PaP-P downregulates ODC activity in H157 cells (72 h treatment with PaP-P, mean of 2 experiments performed in triplicate, +/−SEM). PaP-B/miR-34a (w/w 10, 200 nM miRNA, 48 h) (b) upregulate SMOX and SSAT (by RT-PCR) and (c) deplete Spd and Spm (by HPLC) in HCT116 cells (mean±SD). *p<0.05, **p<0.01, ***p<0.001

FIG. 4 shows in vitro activity of PaP-B/miR34a. (a) Delivery of miR-34a by the PaP-B nanoparticles. RT-PCR analysis of miR-34a levels in HCT116 cells (4 h incubation, 200 nM miRNA). (b) PaP/miR-34a treatment downregulates Bcl-2 (w/w 10, 200 nM miRNA, 48 h). (c) Combination cell-killing effect of PaP-B/miR34a determined by CellTiterBlue viability assay (48 h, 200 nM miRNA). (d) Cell morphology after treatment with PBS, PaP/miR-NC, and PaP/miR-34a (w/w 10, 200 nM miRNA, 48 h, 40×). [(*p<0.05, ***p<0.001) vs. miR-NC

FIG. 5 shows in vivo antitumor activity of PaP-B/miR-34a nanoparticles. (a) Tumor growth inhibition by the nanoparticles in HCT116 tumor xenograft following intratumoral injection of saline (untreated), PaP-B/miR-NC (non-coding control miRNA), and PaP-B/miR-34a nanoparticles (w/w 10, 4 doses, 0.5 mg/kg miRNA). (b) Effect of the treatment on the animal body weight. (c) Downregulation of Bcl-2 by the PaP/miR-34a particles in the tumors. (d) Upregulation of polyamine catabolic enzymes by the PaP/miR-34a and PaP/miR-NC particles in the tumors (RT-PCR). (e) Enhanced miR-34a levels in the tumors following treatment with PaP/miR-34a nanoparticles (miR-34a quantified by qRT-PCR in tumor tissue lysates). (f) Effect of the PaP/miRNA treatments on the content of proliferating (% of Ki-67 positive) cells in tumors (from IHC analysis). Mean+/−SD (n=5), *p<0.05, ***p<0.001.

FIG. 6 shows enhanced serum stability and improved intravenous delivery by fluorinated and bioreducible core-shell nanoparticles. (a) Particle integrity in serum determined from decrease in FRET (Cy3/Cy5) signal vs 0% FBS. (b) Luciferase silencing in 4T1-Luc breast tumors following i.v. injection of saline (PBS) or fluorinated bioreducible nanoparticles prepared with luciferase siRNA (PEG-(F)-RHB/siLuc) or control scrambled control siRNA (PEG-(F)-RHB/siScr). Tumor Luc expression measured immediately before injection (Day 0) and then 2 days after injection (Day 2)—mean Luc tumor expression ±SD (n=3), ***p<0.005). (c) Colloidal stability in PBS. Size measured by DLS. (d) Improved particle size control by microfluidic assembly (green) (75.5 and 73.8 nm) vs. manual pipetting (red) (159.1 and 121.6 nm) using 300 μL/min flow rate and 100 and 75 μg/mL polymer concentration.

FIG. 7 shows iRGD peptide covalently conjugated to the surface of nanoparticles (NP) increases penetration of NP (red) in tumor spheroids and in orthotopic tumors in vivo. NP penetration in spheroids in vitro and tumor in vivo (red: fluorescently labeled polyester NP, green: tumor blood vessels, CD31). iRGD+NP: mixture of iRGD peptide with NP, iRGD−NP: NP with covalently attached iRGD. (48 h after i.v. injection).

FIG. 8 shows (A) PaP condenses miRNA into nanoparticles by electrostatic interactions. (B) Upon endocytosis and endosomal escape, the particles are subjected to cytoplasmic reduction by GSH, which leads to particle disassembly and release of BENSpm and miR-34a mimic. BENSpm induces expression of polyamine catabolic enzymes, which depletes intracellular polyamine levels. MiR-34a mimic increases cellular miR-34a levels, which leads to downregulation of Bcl-2 and upregulation of p53, resulting in enhanced cancer cell death.

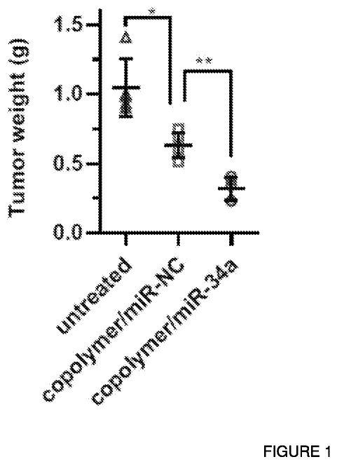

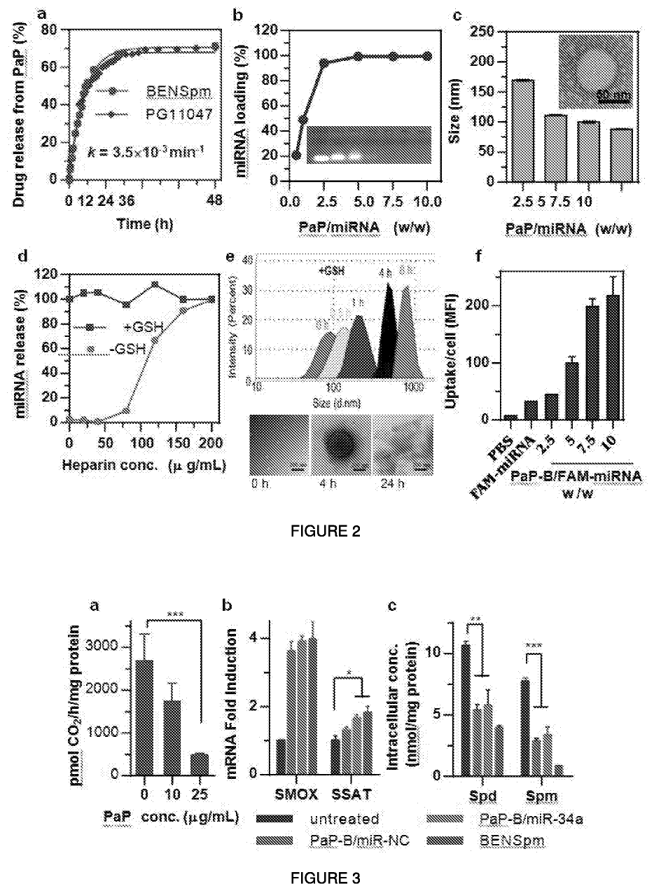

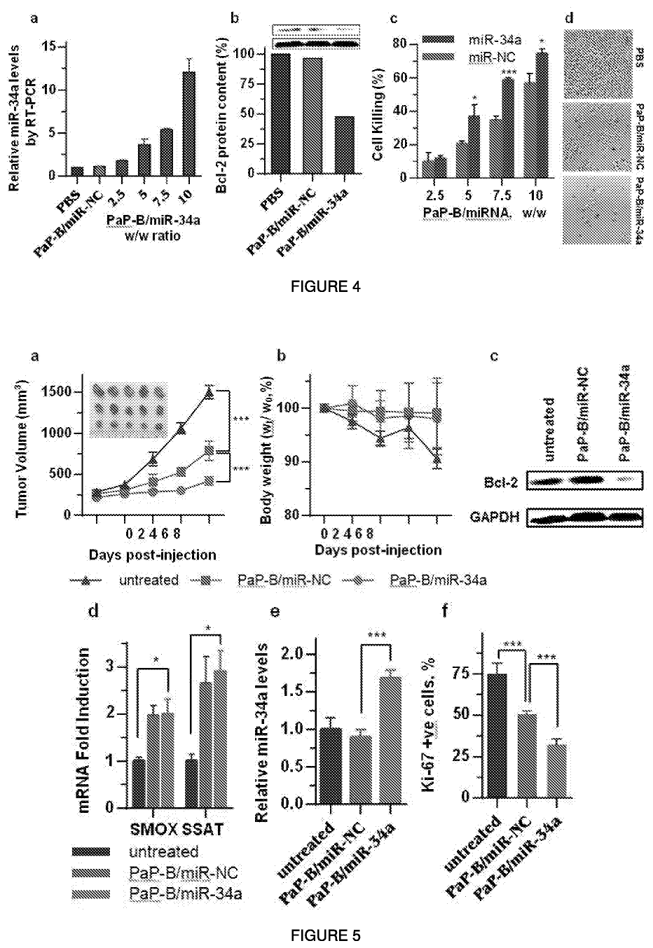

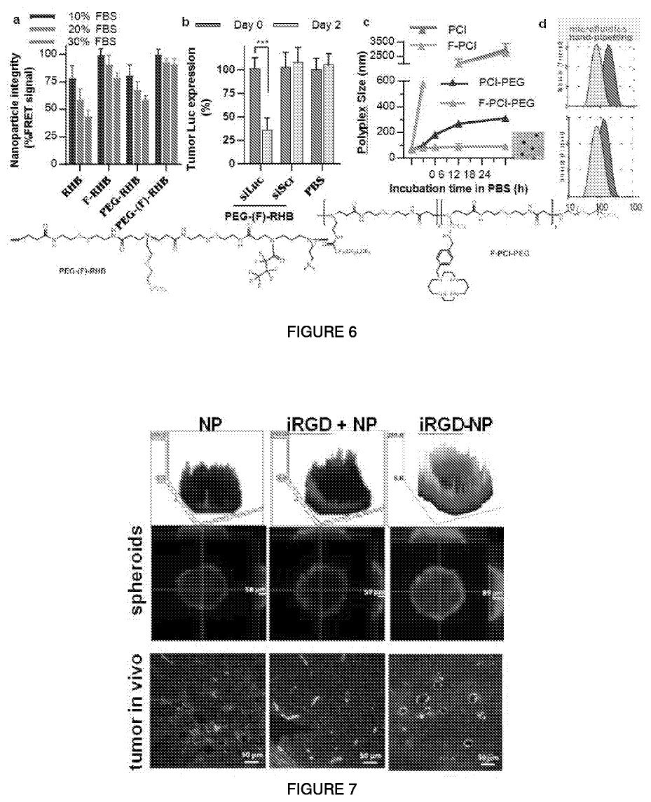

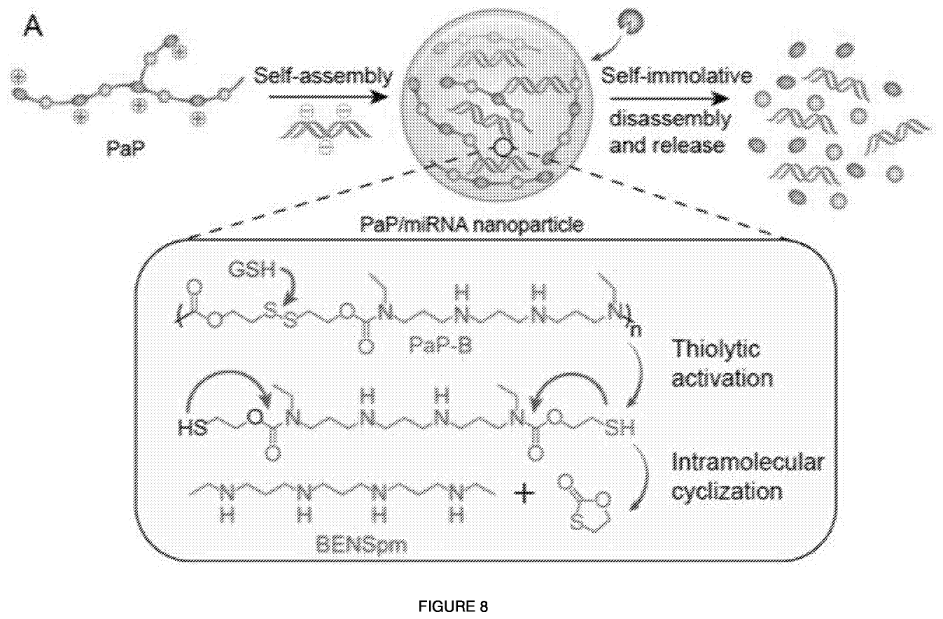

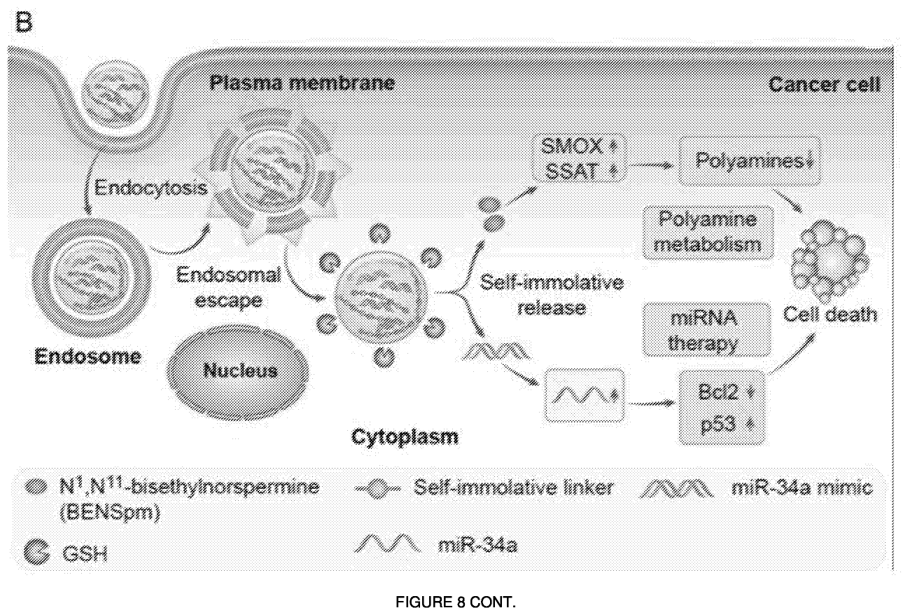

### DETAILED DESCRIPTION

The challenges of RNA or DNA delivery can be addressed with an integrated therapeutic platform that delivers a nucleic acid-based therapy and then degrades into small molecules that modulate dysregulated polyamine metabolism in order to boost the therapeutic activity of the nucleic acid. The use of polyamine copolymers in the development of therapeutics has been described e.g., in PLoS One. 2017; 12(4): e0175917 and J Control Release. 2017 Jan. 28; 246:110-119, the contents of each of which are incorporated herein by reference in their entireties.

Thus, provided herein are copolymers comprising polyamine compounds and degradable linkers that can treat or prevent a disease or disorder associated with dysregulated polyamine metabolism in a subject. These copolymers are useful in the treatment of a variety of diseases and disorders, including but not limited to, Snyder Robinson Disease and cancer.

### Copolymers of the Disclosure

Provided herein are novel compositions and methods for the treatment of cancer. The compositions of this invention are related to polyamine prodrug copolymers and their formulations with nucleic acids including but not limited to microRNA, siRNA, shRNA, DNA, mRNA, IncRNA. The copolymers of the present invention are based on agents called polyamine analogs that target dysregulated polyamine metabolism in cancer. The copolymers contain polyamine prodrugs interspaced by a degradable linker. Without wishing to be bound by theory, cleavage of the degradable linker in the cancer cells leads to the breakdown of the copolymers and release of the parent polyamine analog.

The disclosure provides copolymers comprising monomers of (1) a polyamine and (2) a degradable linker, and further comprising a stabilizing moiety, a fluorinated moiety, a poly(ethylene glycol) (PEG) which is optionally substituted with a targeting moiety, or a combination thereof. In some cases, the copolymer is branched. In some cases, the copolymer comprises alternating monomers of polyamine and degradable linker (e.g., an alternating copolymer). The stabilizing moiety, fluorinated moiety, and/or optionally substituted PEG can be appended to a terminal monomer of the copolymer, or to a monomer within the copolymer. In some cases, where more than on moiety is present in the copolymer, one moiety is appended to one terminus of the copolymer and another moiety is appended to the other terminus of the copolymer.

A variety of polyamines can be used to form the copolymers as described herein. In some embodiments, the copolymer comprises a single polyamine. In other embodiments, the copolymer comprises more than one type of polyamine. Contemplated polyamines include, but are not limited to, (N¹,N¹¹)-bis(ethyl)norspermine (BENSpm), (N¹,N¹²)-bis(ethyl)spermine (BESpm), (N¹,N¹²)-bis(ethyl)-cis-6,7-dehydrospermine (PG-11047), N-[2-aminooxyethyl]-1,4-diaminobutane (AOE-PU), 1-aminooxy-3-aminopropane (APA), 1-aminooxy-3-N-[3-aminopropyl]-aminopropane (AP-APA), 1,3-bis(2-chloroethyl)-1-nitrosourea (BCNU), N¹,N⁸-bis(ethyl)spermidine (BES), CPENSpm, CHENSpm, SL11144, BEHSpm, IPENSpm, 1,12-dimethyl spermine, and PENSpm. In some specific embodiments, the copolymer comprises PG-11047, BENSpm, or a combination thereof.

A variety of degradable linkers can be used in the disclosed copolymers. Contemplated degradable linkers including, but are not limited to, degradable linkers that can be degraded by reducing agents in the cell (e.g., disulfide linkers, sterically hindered disulfide linkers), degradable linkers that can be degraded by elevated levels of reactive oxygen species in tumors, degradable peptide linkers that can be cleaved by enzymes (e.g. Cathepsins, MMPs, esterases) and degradable linkers that are affected by changes of pH (e.g., phosphoramidate linkers, hydrazone linkers, citraconic acid- and dimethylmaleic acid-based linkers). In some embodiments, the degradable linker comprises bis(2-hydroxyethyl)disulfide (BHED).

The copolymers disclosed herein comprise a stabilizing moiety, a fluorinated moiety, a poly(ethylene glycol) (PEG) which is optionally substituted with a targeting moiety, or a combination thereof. In some cases, the copolymer comprises at least two of a stabilizing moiety, a fluorinated moiety, and a PEG optionally substituted with a targeting moiety.

The copolymers described herein modified with fluorinated moiety (or interchangeably termed a substituent), without being bound by theory, can drive assembly of the copolymers into stable lipophobic particles. Contemplated fluorinated moieties include a fluorinated benzoic acid derivative, perfluoroacyl derivatives [CH₃(CF₂)ₙ—CO; CH₃(CF₂)ₙCH₂—CO; CF₃(CF₂)nCH₂—CO;- where n could be 0-6).

The copolymers can be modified with stabilizing moiety including but not limited to fatty acids and cholesterol. In some cases, the fatty acid is a C2-20 fatty acid. In some cases, the fatty acid is perfluorinated or partially fluorinated.

The copolymers can be modified with poly(ethylene glycol) (PEG). The PEG can be attached to a variety of targeting moieties to enhance tumor delivery of the compositions. Targeting moieties include but are not limited to peptides, proteins, and antibodies, which associate with a target of interest. In some cases, the targeting moiety comprises a cyclic peptide. Specific examples of specific targeting moieties include but are not limited to an iRGD peptide, or an analog thereof, folic acid, an EGF-binding peptide, and a HER2 antibody.

Some specifically contemplated copolymers described herein have the structure

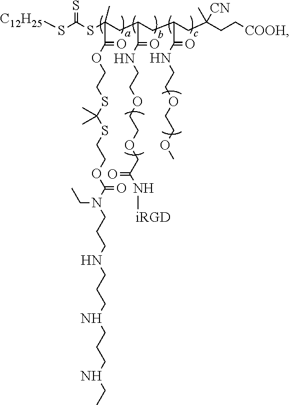

wherein a is from 2-20 mol %, b is 0.5-5 mol %, and c is 75-98.5 mol % (based on a molecular weight of 3-50 kDa);
the structure

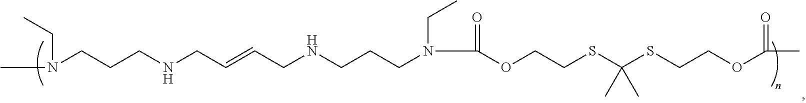

wherein n is 5-100, optionally 4-50; or the structure

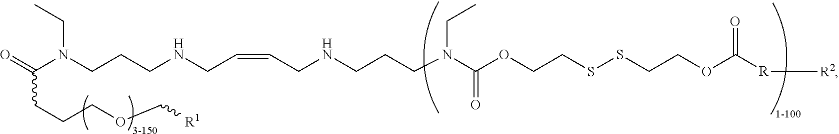

wherein

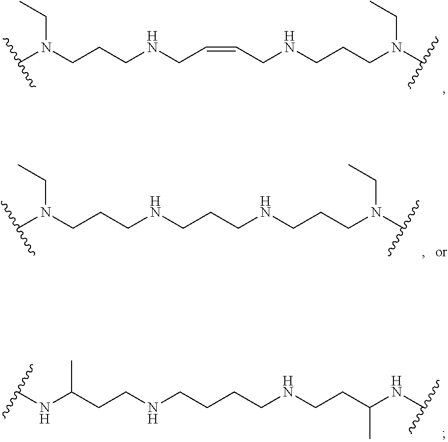 [SMILES: *N(CC)CCCNC/C=C\CNCCCN(*)CC ; *N(CC)CCCNCCCNCCCN(*)CC ; *NC(C)CCNCCCCNCCC(C)N*]

### R is

R¹ is

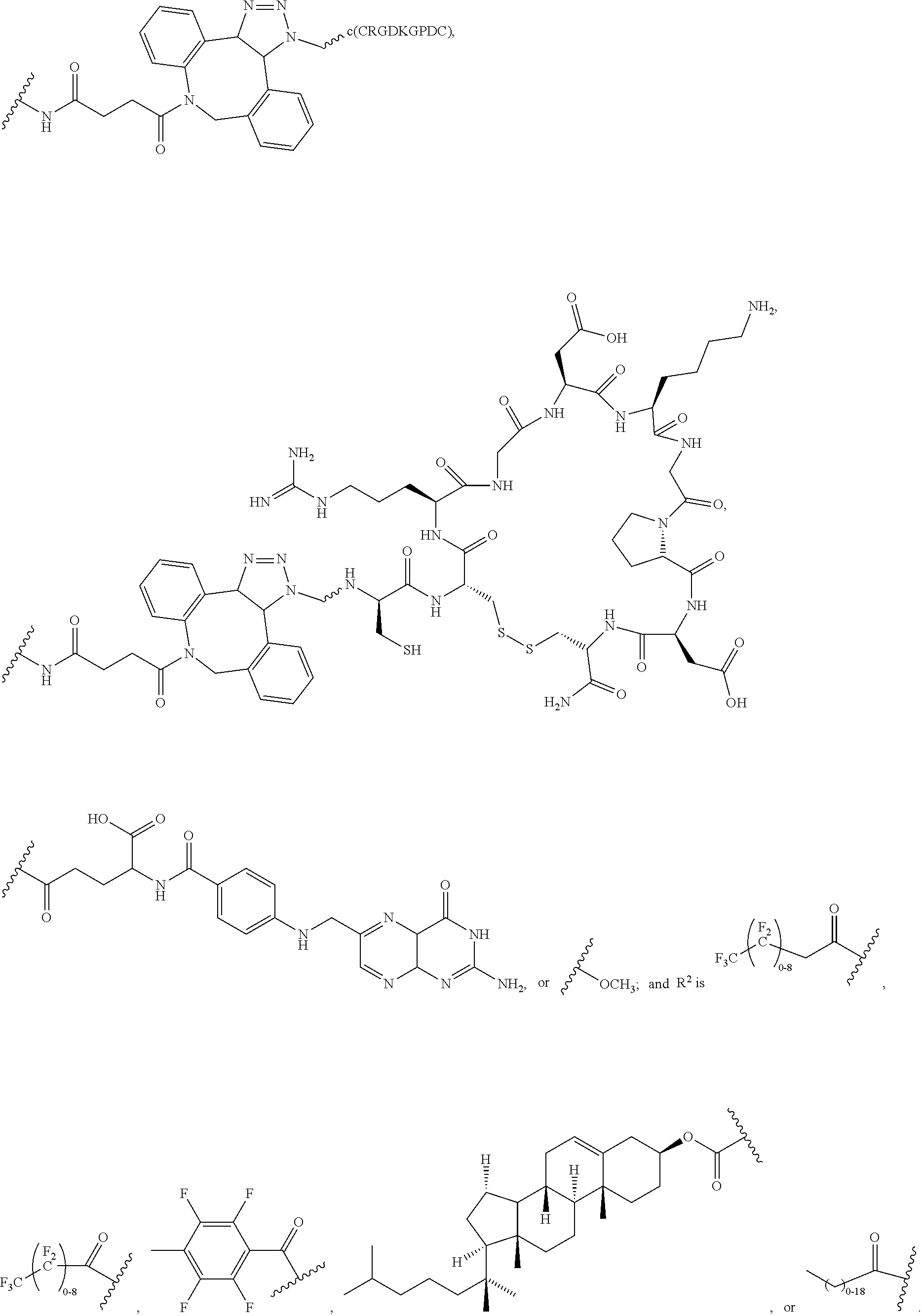

In some cases, the polymer has the structure

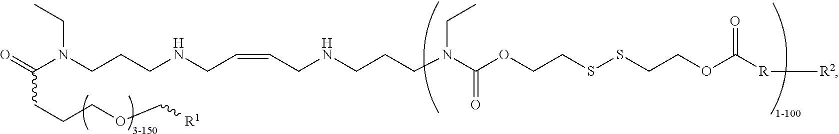

wherein R,
R¹,and R² are each as defined above.

### Nanoparticles of the Disclosure

Nanoparticles can be formed by the copolymers described herein and can optionally further include an oligonucleotide. Without wishing to be bound by theory, copolymers of the present disclosure can bind any negatively charged oligonucleotide and assemble into nanoparticles. The nanoparticles can a plurality of copolymers described herein. In some cases, the oligonucleotide is encapsulated in the nanoparticle.

These nanoparticles can deliver the oligonucleotides to a cell of interest, e.g., a cancer cell, where the copolymers can be degraded into parent drug and the oligonucleotide is released to provide synergistic enhancement of oligonucleotide activity. Oligonucleotides include but are not limited to microRNAs, siRNAs, shRNAs, DNAs, cDNAs, DNA antisense oligonucleotides, DNA aptamers, RNA decoys, circular RNAs, IncRNAs, and mRNAs. In some cases, the oligonucleotide comprises miR34a, miR21, miR210, miR29b, miR200c, or let7.

The use of nanoparticle delivery methods can overcome stability challenges and minimize off-target effects by restricting systemic distribution and improving selectivity of tumor delivery of therapeutics. Self-assembled nanoparticles (polyplexes) based on polyelectrolyte complexes between polycations and miRNA represent one of the most promising approaches. However, because of the dynamic nature of the assembled particles, they are susceptible to disassembly by various competing biomacromolecules in the systemic circulation or at glomerular basement membrane in the kidneys by the action of heparan sulfate. Many solutions have been proposed to the premature systemic disassembly, including modifications with hydrophobic residues and covalent crosslinking of the particles.

While hydrophobic stabilization is a simple method, it suffers from potential disruption with serum lipids and lipoproteins. It was recently proposed that partially fluorinated polycations may overcome many of the issues of hydrophobically modified polycations. Fluorination imparts not only hydrophobic but importantly also lipophobic characteristics and results in a strong tendency towards phase separation in both polar and nonpolar environments. These unique features of the fluorous interactions can improve stability of self-assembled systems against both competing natural polyelectrolytes such as heparin as well as lipids and lipoproteins in vivo. Fluorination can also improve the ability of polycations to transport nucleic acids across the cellular lipid bilayers and thus improve their cytoplasmic delivery by facilitating endosomal escape. Fluorination provides self-assembled nucleic acid particles with excellent serum resistance and leads to dramatically improved transfection. However, recent results indicate the high affinity of the fluorous interactions may compromise the ability of the assembled particles to release the delivered nucleic acids in the intracellular environment. Combining fluorination and bioreducibility leads to particles with the desired high extracellular stability and easy intracellular disassembly and release of miRNA.

miRNAs are a class of small noncoding RNAs that post-transcriptionally regulate gene expression. MiRNAs regulate a wide range of pathways and mediate the expression of nearly 30% of all human proteins. Dysregulated miRNAs in cancer function both as tumor suppressors or oncogenes and play an important role in tumorigenesis, tumor growth, angiogenesis, and metastasis. By targeting different genes at the same time, a single miRNA enables regulation of multiple biological pathways that are critical for a cancer phenotype.48 Thus, restitution of downregulated tumor-suppressor miRNA or inhibition of oncogenic miRNA provides a promising approach to treat cancer. Both miRNA mimics and molecules targeted at miRNAs (anti-miRs) have shown preclinical promise. Several miRNAs have reached clinical development, including a mimic of the tumor suppressor miR-34a. As tumor suppressor, miR-34a is downregulated in a variety of cancers and is an important downstream transcriptional target of the p53 tumor suppressor gene. In developing cancers, miR-34a expression can be lost due to the loss of functional p53 by deletion or mutation, the loss of the miR-34a loci on chromosome 1p, and through epigenetic silencing of the promoter regulating miR- 34a transcription. As miR-34a regulates the expression of other important tumor-associated genes, including Bcl-2, CCND1, CDK4, CDK6, MET, c-MYC, N-MYC, SIRT1, and Notch1, delivery of miR-34a to restore its levels in tumors with low or no expression has an antitumor effect. Moreover, recent reports also show chemosensitization by miR-34a mimics in colon, liver, lung, and breast cancer. Due to the heterogeneity of cancer and the involvement of multiple gene mutations during tumorigenesis and tumor progression, combining miR-34a mimics with other treatments has a potential to target multiple cancer-associated pathways and overcoming drug resistance.

The nanoparticles as disclosed herein can be used to treat a variety of diseases including cancer and other diseases that have dysregulated polyamine metabolism such as Snyder Robinson Disease (also called Snyder-Robinson X-linked mental retardation syndrome).

The nanoparticles can be used as a gene therapy delivery system. Genes can be packaged into the copolymers to form a nanoparticle capable of delivering the gene to desired cells and/or tissues either systemically or by targeting the nanoparticles to specific tissues and/or cells using targeting ligands. The genes can be packaged in a vector system or may be unpackaged (ex. DNA or cDNA). In some embodiments, the nanoparticles are used to deliver the spermine synthase gene. The spermine synthase gene can be present as cDNA. A nanoparticle comprising a spermine synthase gene can be used, e.g., to treat Snyder Robinson Disease.

The nanoparticles disclosed herein can be used to treat a variety of diseases including cancer and other diseases that have dysregulated polyamine metabolism such as Snyder Robinson Disease (also called Snyder-Robinson X-linked mental retardation syndrome).

### Synthesis of Polyamine Copolymers of the Disclosure

The copolymers disclosed herein can be prepared in a variety of ways using commercially available starting materials, intermediates as reported in the literature, or from readily prepared intermediates, by employing standard synthetic methods and procedures either known to those skilled in the art, or in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March' s Advanced Organic Chemistry: Reactions, Mechanisms, and Structure, 5^{th} edition, John Wiley & Sons: New York, 2001 ; and Greene, T. W., Wuts, P. G. M., Protective Groups in Organic Synthesis, 3^{rd} edition, John Wiley & Sons: New York, 1999, are useful and recognized reference textbooks of organic synthesis known to those in the art. The following descriptions of synthetic methods are designed to illustrate, but not to limit, general procedures for the preparation of compounds and copolymers of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt, ester or prodrug thereof.

In general, copolymers as described herein can be synthesized according to Scheme 1.

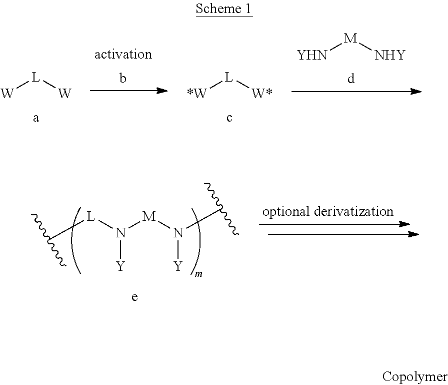

Copolymers having structure e can be synthesized using the procedure shown in Scheme 1. Reaction of a degradable linker monomer a with an activating reagent b produces an activated degradable linker monomer, which can optionally be isolated (structure c). In Scheme 1 above, each “W” represents a functional group, such as a hydroxyl group, that can be activated with a suitable reagent, e.g., 1,1′-carbonyldiimidazole (CDI), and “L” represents the remaining portion of the monomer. Coupling of the activated degradable linker monomer c with a polyamine monomer d provides polyamine copolymers e as described herein. In Scheme 1 above, “M” represents the portion of the polyamine monomer other than the amine moieties which react with the activated degradable linker monomer, and each Y is independently H or a non-H substituent, such as an alkyl group. Optional subsequent derivatization gives compounds as described herein, i.e., copolymers comprising a stabilizing moiety, a fluorinated moiety, a poly(ethylene glycol) (PEG) which is optionally substituted with a targeting moiety, or a combination thereof. Appropriate derivatization reactions can be selected based on the nature of residues L and M.

The coupling of compounds c and d can be catalyzed by appropriate reagents selected based on the precise nature of compounds c and d. For example, when compound c is a dihydroxyl compound (i.e., when each W is OH), the coupling of compounds c and d can be catalyzed by e.g., CDI. Occasionally, the coupling reaction may not require a catalyst, e.g., when compound a is a diacyl chloride (i.e., when each W is C(O)Cl).

Compounds a and c can be purchased commercially or prepared by a variety of methods from commercially-available starting materials. For example, diamines such as compounds having structure d can be prepared by the reduction (e.g., palladium-catalyzed reduction) of appropriate compounds bearing nitro functional groups. Dialdehyde compounds having structure a can be prepared by the reduction (e.g., borohydride reduction) of appropriate compounds bearing carbonyl functional groups.

Derivatization reactions to transform compounds having structure e into copolymers comprising a stabilizing moiety, a fluorinated moiety, a poly(ethylene glycol) (PEG) which is optionally substituted with a targeting moiety, or a combination thereof can be selected based on the nature of the residues L and M in copolymer e and the functionality desired. For example, derivatization with PEG groups can be effected using coupling reagents known in the art, e.g., when L or M comprises a carboxylic acid moiety, carbodiimide chemistry (such as a Mitsunobu reaction) can be used to link a PEG moiety to the compound.

Additional synthetic procedures for preparing the compounds disclosed herein can be found in the Examples section.

### Methods of Use

The copolymers disclosed herein can be used in many different therapeutic applications, such as treatment of Snyder Robinson Disease or cancer.

The present disclosure provides a method of delivering a therapeutic to a cell comprising contacting the cell with the nanoparticle comprising a polyamine copolymer described herein, wherein upon contact with the cell, the nanoparticle releases the therapeutic into the cell, e.g. a gene. In some cases, the method comprises contacting the cell with the nanoparticle, wherein upon contact with the cell, the nanoparticle releases the gene into the cell. In some cases, the cell comprises tissue and the copolymer of the nanoparticle comprises a targeting moiety specific to the tissue. In some cases, the contacting is in vivo. In some cases, the contacting comprises administering to a subject in need thereof and the administration results in treatment of a disease or disorder associated with aberrant activity of the gene.

The present disclosure also provides a method for treating a disease associated with dysregulated polyamine metabolism in a patient by administering to the patient a therapeutically effective amount of a pharmaceutical composition comprising a polyamine copolymer of the present disclosure In some cases, the disease is Snyder Robinson Disease.

The present disclosure also provides a method for treating cancer in a patient by administering to the patient a therapeutically effective amount of a pharmaceutical composition comprising a polyamine copolymer of the present disclosure. In some cases, the polyamine copolymer comprises miRNA useful for the treatment of a disease or disorder, e.g., cancer. In some cases, the pharmaceutical composition comprises miR34a, miR21, miR210, miR29b, miR200c, or let7.

In some cases, the cancer is lung cancer, lymphoma, prostate cancer, breast cancer, or colon cancer. In some cases, the cancer is lung cancer. In some cases, the lung cancer is non-small cell lung cancer.

In some cases, the pharmaceutical composition is administered with a chemotherapeutic agent. In some cases, the chemotherapeutic agent is an HDAC inhibitor, cisplatin, erlotinib, 5-fluorouracil, or bevacizumab, or a combination thereof.

### EXAMPLES

The following examples are provided for illustration and are not intended to limit the scope of the disclosure.

### Synthetic Procedures for Copolymers

General Experimental Procedures. All reagents and solvents were obtained from commercial sources and used without additional purification.

### Example 1—Synthesis of ROS-Methacrylate Polymers

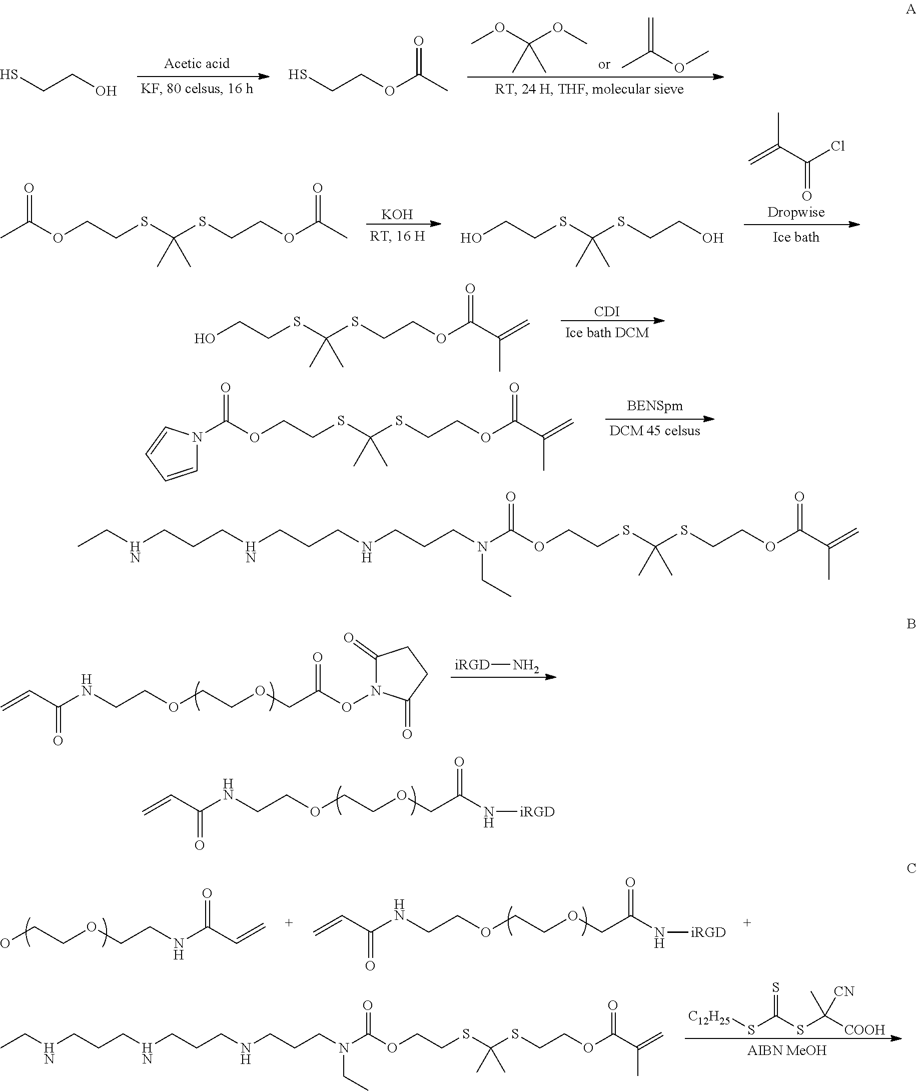

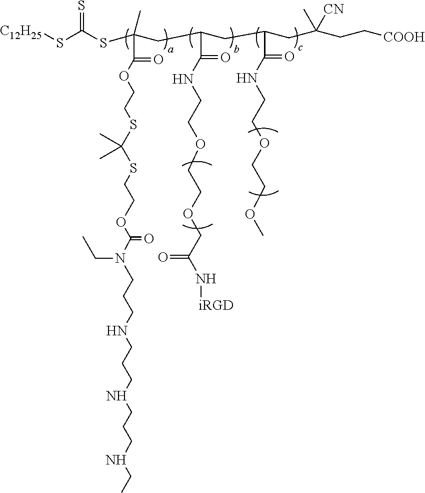

Synthesis of acetyl protected mercaptoethanol. Mercaptoethanol (4.84 g, 62 mmol) and KF (4.4 g, 76 mmol) were suspended in 100 mL of acetic acid and the mixture was stirred at 80° C. for 16 h. Subsequently, the mixture was diluted with deionized water, and the crude product was extracted with ethyl acetate. After multiple washings with saturated sodium bicarbonate and brine, the organic phase was dried over Na₂SO₄. The purified product was obtained as liquid after passing flash silica gel column.

Synthesis of acetyl protected thioketal linker. 2,2-Dimethoxypropane (0.42 g, 4 mmol), acetyl protected mercaptoethanol (1.2 g, 10 mmol), and PTSA (0.57 g, 5 mmol) were dissolved in 25 mL of toluene. After addition of 5 Å molecular sieves (10 g), the mixture was stirred at room temperature for 24 h. Subsequently, the sieves were removed and the solvent was evaporated to give the crude product, which was then purified via column chromatography using a gradient mobile phase from 100% hexane to 50% hexane and 50% ethyl acetate.

Synthesis of thioketal linker. Acetyl protected thioketal linker (1.17 g, 4 mmol) and KOH (1.0 g, 18 mmol) were suspended in 15 mL methanol, and the mixture was stirred at room temperature for 16 h. Thereafter, the solvent was removed by rotary evaporator, and the residues were dissolved in deionized water. After adding hydrochloric acid solution to neutralize the solution pH, ethyl acetate was added to extract the product. After drying over Na₂SO₄ and removal of the solvent, the thioketal linker was collected as liquid.

Synthesis of MA-thioketal linker. Thioketal linker (1.2 g, 5.95 mmol) and triethylamine (0.59 g, 0.73 mL, 0.31 mmol) were dissolved in chloroform (50 mL) and cooled down in the ice bath. Methacryloyl chloride (0.67 g, 0.56 mL, 5.80 mmol) was dissolved in anhydrous chloroform (50 mL) and added to the HCQ dropwise with vigorously stirring at 0° C. The mixture was stirred overnight, followed by washing with saturated sodium carbonate (2×50 mL) and brine (50 mL). The resulted organic layer was concentrated and purified by silica gel chromatography (10:1 dichloromethane:methanol) to give the MA-thioketal linker.

Synthesis of MA-thioketal-PG11047. MA-thioketal linker were dissolve in DCM and tetrahydrofurane (THF). and CDI were suspended in DCM. The thioketal linker solution was added in CDI suspension dropwise under nitrogen protection and ice bath for 1 h. The excessive CDI is quenched by adding 5 ml water. Subsequently, the water layer is removed via separation and DCM layer is dried with sodium sulfate.

Modification of RAFT agent. 4-Cyano-4-[(dodecylsulfanylthiocarbonyl)sulfanyl]pentanoic acid and EDC.HCl were dissolved in anhydrous chloroform and stirred at room temperature for 30 min. The mixture was cooled down in ice bath followed by addition of DBCO-PEG-amine dissolved in chloroform. After stirring overnight, the resulting product was concentrated and purified by column chromatography with DCM as eluent.

RAFT polymerization. MA-thioketal-PG11047, modified RAFT agent and AIBN were dissolved in 1:1 mixture of 1,4-dioxane and DMSO (100 mg/mL) and purged by argon for 30 min. After stirring in a flame-sealed ampule at 70° C. for 16 h, the reaction was terminated in liquid nitrogen and the polymer was precipitated by cold diethyl ether under vigorous stirring. The precipitates were collected and re-dissolved in DMF. The precipitation step was repeated twice and the polymers then were dialyzed against water (MWCO: 3,500) for 2 days. To prepare iRGD modified polymer, azido-functionalized iRGD was added to polymer aqueous solution. After reaction at 37° C. for 30 mins, the polymers were dialyzed against water for 2 days.

### Example 2—Decoration of ROS-Methacrylate Polymers with Targeting Moiety

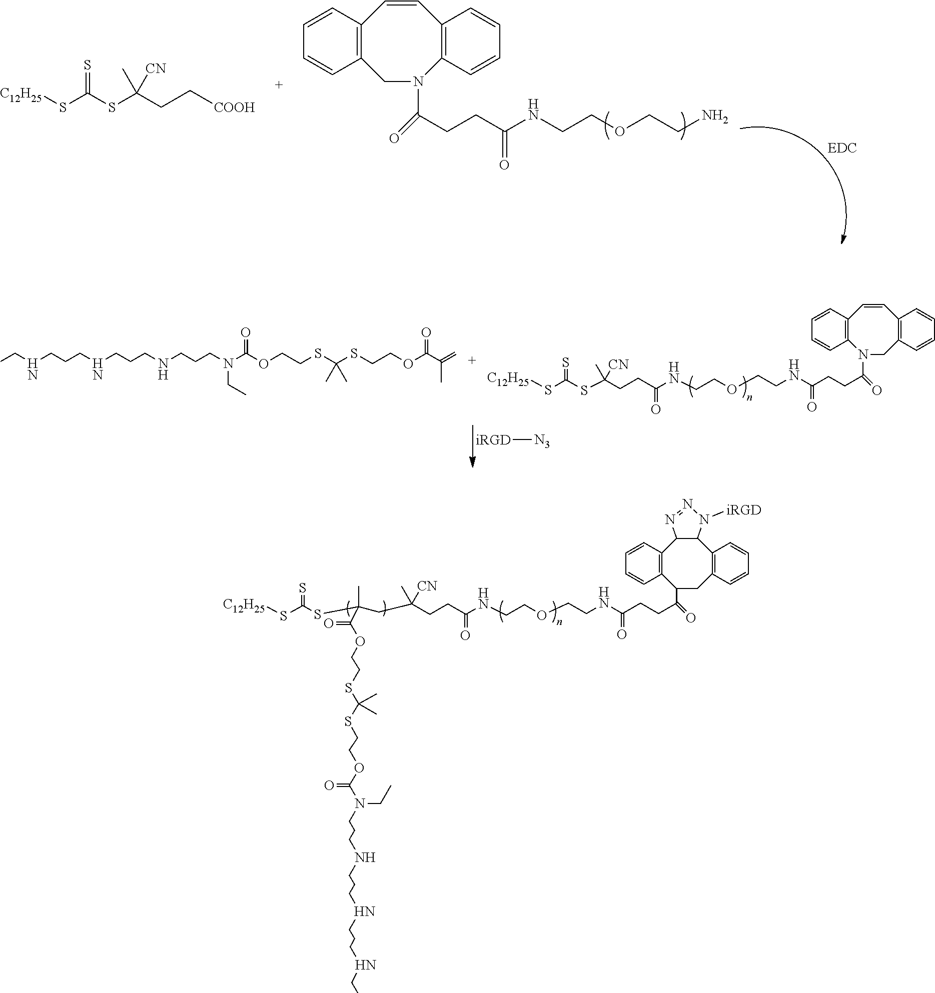

For iRGD-conjugation, azido functionalized iRGD is added to aqueous solution of the polymer with dibenzocyclooctyne group (DBCO). After 30 min reaction at 37° C., the iRGD-modified polymer or nanoparticles are collected.

### Example 3—Synthesis of ROS Step Polymers

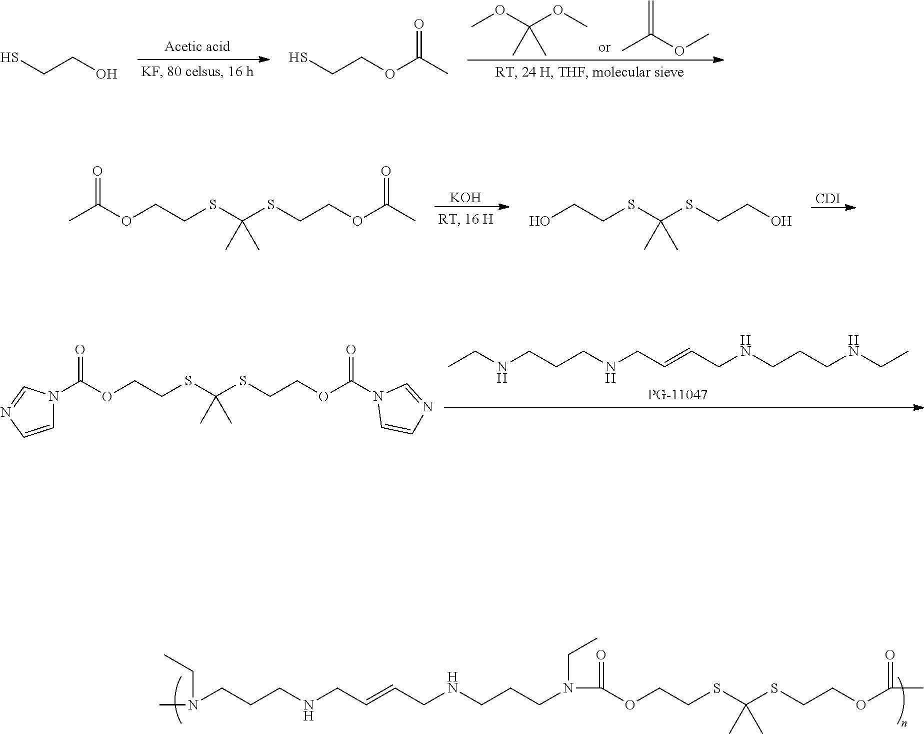

Synthesis of acetyl protected mercaptoethanol. Mercaptoethanol (4.84 g, 62 mmol) and KF (4.4 g, 76 mmol) were suspended in 100 mL of acetic acid and the mixture was stirred at 80° C. for 16 h. Subsequently, the mixture was diluted with deionized water, and the crude product was extracted with ethyl acetate. After multiple washings with saturated sodium bicarbonate and brine, the organic phase was dried over Na₂SO₄. The purified product was obtained as liquid after passing flash silica gel column.

Synthesis of acetyl protected thioketal linker. 2,2-Dimethoxypropane (0.42 g, 4 mmol), acetyl protected mercaptoethanol (1.2 g, 10 mmol), and PTSA (0.57 g, 5 mmol) were dissolved in 25 mL of toluene. After addition of 5 Å molecular sieves (10 g), the mixture was stirred at room temperature for 24 h. Subsequently, the sieves were removed and the solvent was evaporated to give the crude product, which was then purified via column chromatography using a gradient mobile phase from 100% hexane to 50% hexane and 50% ethyl acetate.

Synthesis of thioketal linker. Acetyl protected thioketal linker (1.17 g, 4 mmol) and KOH (1.0 g, 18 mmol) were suspended in 15 mL methanol, and the mixture was stirred at room temperature for 16 h. Thereafter, the solvent was removed by rotary evaporator, and the residues were dissolved in deionized water. After adding hydrochloric acid solution to neutralize the solution pH, ethyl acetate was added to extract the product. After drying over Na₂SO₄ and removal of the solvent, the thioketal linker was collected as liquid.

Synthesis of CDI-activated thioketal linker. The thioketal linker (77 mg, 0.39 mmol) were dissolve in 0.60 ml DCM and 0.12 ml tetrahydrofurane (THF). and CDI (162.2 mg, 1 mmol) were suspended in 1.4 ml DCM. The thioketal linker solution was added in CDI suspension dropwise under nitrogen protection and ice bath for 1 h. The excess CDI was quenched by adding 5 ml water. Subsequently, the water layer is removed via separation and DCM layer was dried with sodium sulfate, followed by evaporation.

Step polymerization with PG-11047. The CDI-activated thioketal linker (113 mg, 0.29 mmol) and PG-11047 (74 mg, 0.29 mmol) were mixed in 2 ml DMF react under nitrogen protection at 40° C. for 18 h. The mixture was then added to 5 ml 10% hydroxyl chloride. The mixture was dialyzed against water for 2 days and freeze dried for 2 days to yield the title product.

### Biological Assay Data

### Example 4—Tumor Inhibition in Mouse Xenograft Colon Cancer Model

Female athymic nude mice (5-6 weeks) were purchased from Charles River Laboratories. The xenograft tumor model was generated by subcutaneous injection of HCT116 cells (5×106 per mouse) into the flank region of the mouse. When the tumor volume reached 200-300 mm3, mice were randomly divided into 3 groups (n=5) and injected intratumorally with 5% glucose, DSS-BEN/miR-NC, and DSS-BEN/miR-34a every other day (total of 4 doses) at doses of 5 mg/kg DSS-BEN and 0.5 mg/kg miRNA (w/w=10). Tumor volume was monitored by measuring the perpendicular size of the tumors using digital calipers. The estimated volume was calculated according to the following formula: tumor volume (mm3)=0.5×length×width 2. Body weight of the mice was also recorded. At the end of the study, mice were sacrificed and all tumor tissues were collected, weighed, and used for further analysis. The total RNA was isolated using mirVana™ miRNA Isolation Kit according to the protocol. Then, the levels of miR-34a and SMOX and SSAT mRNA in the tumors was analyzed by qRT-PCR. For Western blot analysis, tumor tissues were lysed with RIPA Lysis buffer and then were analyzes as described above. For immunohistochemistry analysis, tumors were fixed for 24 h in 4% paraformaldehyde. The tissues were embedded in paraffin and 5 mm sections were cut. The proliferation of tumor cells was determined using the Ki-67 immunohistochemical staining according to the manufacturer's instructions. Images of Ki-67 stained tumor sections were taken by EVOS xl microscope (×40). The number of Ki-67 positive cells and the total number were counted using the ImageJ software. The percentage of Ki-67 positive cells in the samples was obtained by dividing the number of Ki-67 positive cells from the number of total cells in each microscopic field.

In view of the many possible embodiments to which the principles of the disclosure may be applied, it should be recognized that the illustrated embodiments are only examples and should not be taken as limiting the scope of the invention.

1. A copolymer comprising monomers of (1) a polyamine and (2) a degradable linker, and further comprising a stabilizing moiety, a fluorinated moiety, a poly(ethylene glycol) (PEG) which is optionally substituted with a targeting moiety, or a combination thereof.

2. The copolymer of claim 1, wherein the copolymer is branched.

3. The copolymer of claim 1, wherein the copolymer is an alternating copolymer.

4. The copolymer of claim 1, wherein the polyamine comprises (N¹,N¹¹)-bis(ethyl)norspermine (BENSpm), (N¹,N¹²)-bis(ethyl)spermine (BESpm), (N¹,N¹²)-bis(ethyl)-cis-6,7-dehydrospermine (PG-11047), N-[2-aminooxyethyl]-1,4-diaminobutane (AOE-PU), 1-aminooxy-3-aminopropane (APA), 1-aminooxy-3-N-[3-aminopropyl]-aminopropane (AP-APA), 1,3-bis(2-chloroethyl)-1-nitrosourea (BCNU), N¹,N⁸-bis(ethyl)spermidine (BES), CPENSpm, CHENSpm, SL11144, BEHSpm, IPENSpm, 1,12-dimethyl spermine, PENSpm, spermine, or a combination thereof.

5. (canceled)

6. The copolymer of claim 1, wherein the degradable linker comprises a disulfide linker, an enzymatically-cleavable linker, a thioketal ROS-sensitive linker, or a pH-sensitive linker.

7. (canceled)

8. (canceled)

9. The copolymer of claim 1, wherein the degradable linker comprises a phosphoramidate linker, a hydrazone linker, a citraconic acid-based linker, or a dimethylmaleic acid-based linker.

10. The copolymer of claim 6, wherein the enzymatically-cleavable linker is degradable by a cathepsin, MMP, or an esterase.

11. The copolymer of claim 1, wherein the degradable linker comprises bis(2-hydroxyethyl)disulfide (BHED).

12. The copolymer of claim 1 comprising at least two of a stabilizing moiety, a fluorinated moiety, and a PEG optionally substituted with a targeting moiety.

13. The copolymer of claim 1, wherein the stabilizing moiety comprises a fatty acid or cholesterol.

14. (canceled)

15. (canceled)

16. (canceled)

17. (canceled)

18. The copolymer of claim 12, comprising PEG substituted with a targeting moiety, wherein the targeting moiety comprises an iRGD peptide or analog thereof, folic acid, an EGF-binding peptide, or an HER2 antibody.

19. The copolymer of claim 1, comprising a fluorinated moiety, optionally wherein the fluorinated moiety comprises a fluorinated benzoic acid derivative.

20. (canceled)

21. The copolymer of claim 1, having a structure 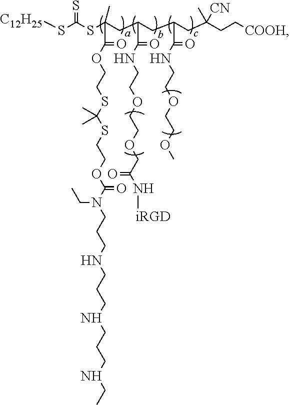
wherein a is from 2-20 mol %, b is 0.5-5 mol %, and c is 75-98.5 mol % (based on a molecular weight of 3-50 kDa);
the structure 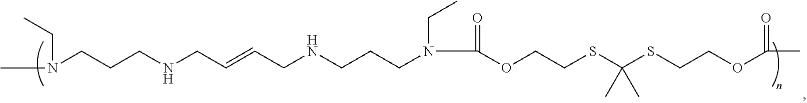
wherein n is 5-100; or the structure 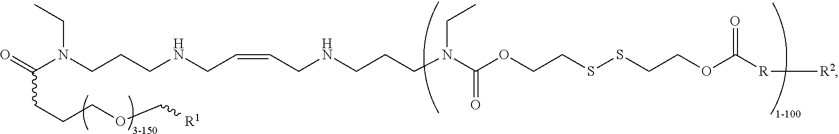
wherein R is 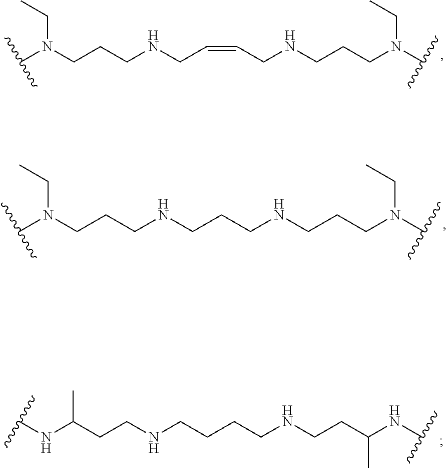 [SMILES: *N(CC)CCCNC/C=C\CNCCCN(*)CC ; *N(CC)CCCNCCCNCCCN(*)CC ; *NC(C)CCNCCCCNCCC(C)N*]
R¹ is 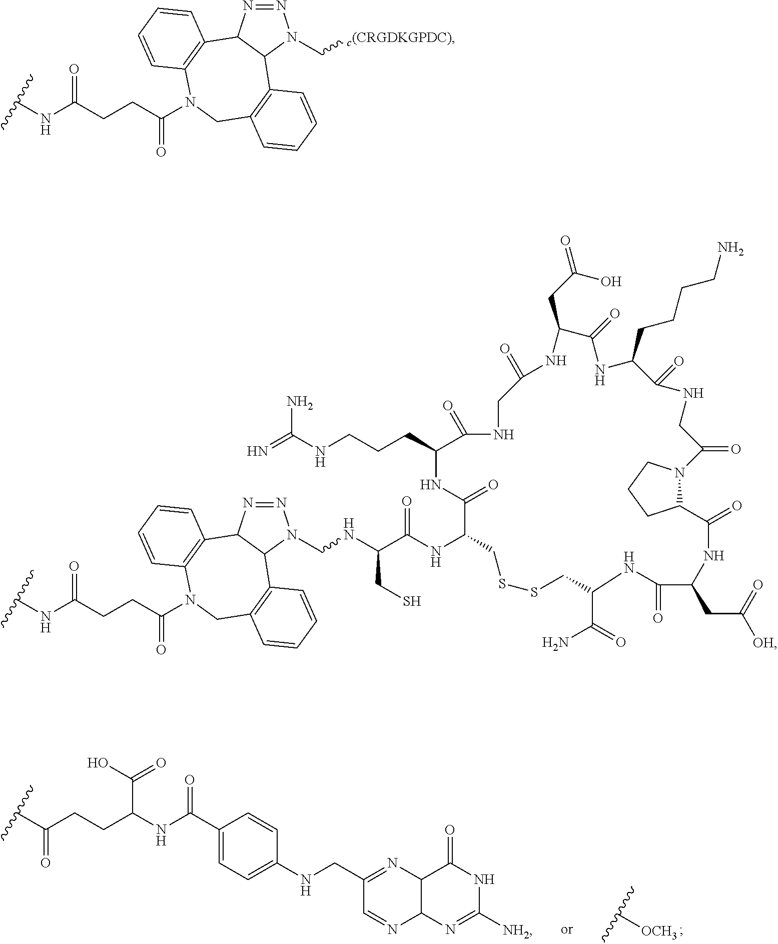
and R² is

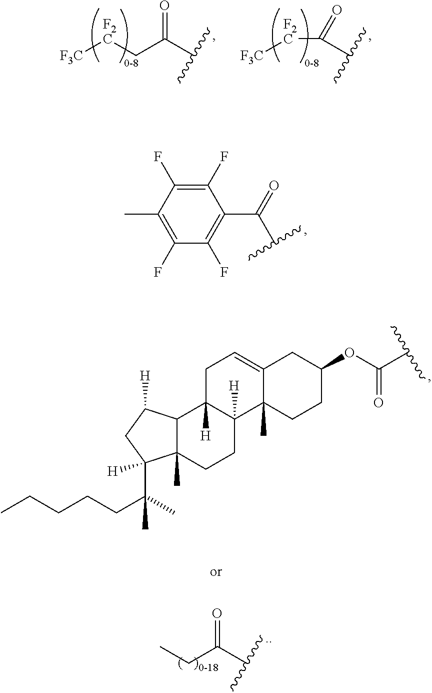

22. A nanoparticle comprising a plurality copolymers of claim 1.

23. (canceled)

24. The nanoparticle of claim 22, further comprising an oligonucleotide, wherein the oligonucleotide is encapsulated in the nanoparticle.

25. The nanoparticle of claim 22, further comprising an oligonucleotide, wherein the oligonucleotide is miRNA, siRNA, shRNA, DNA, cDNA, DNA antisense oligonucleotide, DNA aptamer, RNA decoy, circular RNA, IncRNA, or m RNA.

26. (canceled)

27. (canceled)

28. (canceled)

29. (canceled)

30. The nanoparticle of claim 22, further comprising a gene, wherein the gene comprises a spermine synthase gene.

31. (canceled)

32. A method of delivering a gene to a cell comprising contacting the cell with the nanoparticle of claim 22, wherein the nanoparticle further comprises as genet, and upon contact with the cell, the nanoparticle releases the gene into the cell.

33. (canceled)

34. (canceled)

35. (canceled)

36. (canceled)

37. The method of claim 32, wherein, contacting comprises administering to a subject in need thereof and the administration results in treatment of a disease or disorder associated with aberrant activity of the gene, the disease or disorder is cancer, and the cancer is lung cancer, lymphoma, prostate cancer, breast cancer, or colon cancer.

38. (canceled)

39. The method of claim 37, further comprising administering a chemotherapeutic agent, wherein the chemotherapeutic agent is an HDAC inhibitor, cisplatin, erlotinib, 5-fluorouracil, bevacizumab, or a combination thereof.

40. (canceled)
